(19) United States
(12) Patent Application Publication
(10) Pub. No.: US 2026/0102536 A1
(43) Pub. Date: Apr. 16, 2026

(54) BIOADHESIVE COMPOSITION

(71) Applicants: Institut Straumann AG, Basel (CH); Biomimetic Innovations Limited, Shannon, Co. Clare (IE)

(72) Inventors: Ole ZOFFMANN ANDERSEN, Fredericia (DK); Benjamin PIPPENGER, Hegenheim (FR); Benjamin BELLON PECECNIK, Basel (CH)

(73) Assignees: Institut Straumann AG, Basel (CH); Biomimetic Innovations Limited, Shannon, Co. Clare (IE)

(21) Appl. No.: 19/140,052

(22) PCT Filed: Dec. 20, 2023
(86) PCT No.: PCT/EP2023/087148

§ 371 Date: Jun. 17, 2025

(30) Foreign Application Priority Data
Dec. 21, 2022 (EP) 22215448.6
May 25, 2023 (EP) 23175491.2

(51) Int. Cl. A61L 27/04; A61L 27/36; A61L 27/56; A61L 27/58

(52) U.S. Cl. CPC A61L 27/04; A61L 27/3608; A61L 27/56; A61L 27/58

(57) ABSTRACT

A two-component composition including a first component A including water or an aqueous solution and a second component B including a self-setting adhesive powder including at least a multivalent metal salt and phosphoserine, and at least one hydrogen carbonate, wherein the hydrogen carbonate is present in a concentration between 1 and 8% by weight of component B.

The present invention relates to a composition for preparing an adhesive bone graft material. A kit and a method for preparing said adhesive bone graft material are also provided.

Medical devices such as implants in general and dental implants in particular are widely used nowadays. They have become an appreciated possibility where hard tissue structures need to be repaired or replaced, e.g., in the case of bone fractures or tooth loss. However, the success of such implants strongly depends on adequate support at the implant site. If the bone mass at said site is insufficient or poor in quality, bone repair and/or bone augmentation becomes a necessity. There are different treatments applied to regain sufficient bone mass, the including use of bone graft materials of different origin, shape and size.

While there are ways to systemically treat the mass and/or strength of the bone, e.g., in osteoporosis, it is still difficult to achieve bone formation in a reliable and controllable manner. However, local bone formation would greatly benefit the adequate treatment of incidents where enhancing the bone volume is only required locally, for example, when placing dental implants.

Methods currently used to repair bone defects include graft materials from different sources. The material is either synthetic or of natural origin. One natural graft material which is employed is autogenous bone. In contrast to bone or bone like material from natural sources (human, animals, plants, algae etc.), autogenous bone material does not trigger a strong immune response and is thus not rejected by the host. However, autogenous bone material requires a second surgery for harvesting the bone increasing the risk of unwanted infection and/or inflammation at this site and significantly increases treatment costs. Further, the removal of bone material leads, at least temporarily, to a weakened structure at this site and causes a painful healing process.

EP2269663A2 discloses a bone graft or biocomposite for treating osseous defects and neogenesis of bone. It is a composite of a biodegradable polymer and granules of beta-tricalciumphosphate, and further comprises an active ingredient embedded in the biodegradable polymer.

WO2010056811 discloses compositions comprising at least tetra calcium phosphate, an effective amount of a compound that is structurally similar to phosphoserine, and can be mixed with an aqueous solution. The compositions provide adhesive and cohesive strength in both wet and dry environments.

WO2016196371 and WO2018060289 disclose compositions having adhesion properties and methods of fixation of an implant to a bone. Said materials have excellent adhesion properties, in particular as soft tissue adhesive, however, they are very dense which has a negative impact on new bone formation.

WO2021092062A1 discloses adhesive compositions including a therapeutic that is released from the composition to treat any number of ailments or conditions or to help accelerate local tissue regeneration or to assist with surgical or therapeutic treatment.

The object of the present invention is to provide a bioadhesive composition that allows a good osseointegration.

The problem is solved by the composition according to claim 1. Further preferred embodiments are subject of dependent claims 2 to 15.

The composition of the present invention relates to a two-component composition comprising a first component A and a second component B. The first component A comprises water or an aqueous solution. The second component B comprises a self-setting adhesive powder and at least one hydrogen carbonate. Said self-setting adhesive powder comprises at least a multivalent metal salt and phosphoserine. The at least one hydrogen carbonate is present in a concentration between 1 and 8% by weight of component B.

Upon mixing the two components, i.e. component A and component B, a cohesive, viscous paste is formed, which maintains its tacky character until set. At the same time a gas is created which forms a porous scaffold in said paste. This porous paste can be applied for example to stabilize a implant, in particular a dental implant, or to fill a bone defect. One of the primary advantages of the composition according to the present invention is its inherent ability to set and maintain its adhesive character even in aqueous environments, while at the same time resulting an excellent osseointegration due to the porosity created in situ. This allows for example to adhere a dental implant to bone tissue or soft tissue without having a negative impact on the osseointegration.

It has been found out that the amount of hydrogen carbonate present in the composition according to the present invention is of crucial importance to obtain sufficient porosity in the cured material while at the same time maintaining sufficient stability. For example, a hydrogen carbonate concentration of less than 1% by weight of component B results in a bone graft material which is too dense to guarantee a good osseointegration and a concentration of for example 10% by weight strongly impacts the mechanical stability of the bone graft material. The composition according to the present invention also serves as a mechanically stable scaffold for bone regrowth as well as implant stabilization.

The composition according to the present invention does not only result in an optimal osseointegration by facilitating the ingrowth of vessels and osseous tissue but also serves as a mechanically stable scaffold for these processes. Furthermore, it allows a simple application compared to other materials.

The composition according to the present invention is particularly useful as a bone restorative composition. By a bone restorative composition is meant a composition that is useful to restore and/or repair bone, such as bone adhesives, bone cements, bone glues, bone putties, bone void fillers, bone replacement compositions, cements and/or adhesives to stabilize implants, such as dental implants to bone tissue or soft tissue. The composition according to the present invention is particularly useful in the treatment of bone defects, since it has both, a porous scaffold and adhesive properties. Furthermore, as the material expands during the hardening process, it is especially beneficial to fill complex bone defects. Directly after mixing the composition flows smoothly and homogeneously and expands in the void and fills the complete volume of the defect. Furthermore, as explained above the material has excellent adhesive properties and therefore, allows a good adhesion to the surrounding bone or soft tissue and/or implant materials. Thus, it does not only adhere to biological tissues, but also to materials made of metals, ceramic or plastic, and in particular to dental implants made of titanium, a titanium alloy or Y-TZP.

The first component A of the two-component composition comprises water or an aqueous solution. Within the context of the present invention the term aqueous solution means water that additionally comprises an additive such as a salt, a buffer or the like. Preferably, component A comprises distilled water or an aqueous solution comprising water and saline (0.9% NaCl in water).

The second component B of the two-component composition comprises a self-setting powder. The term “self-setting” refers to the ability of the material to cure and harden as a result of the mixing of the solid and the liquid component. Said self-setting powder comprises at least two different ingredients, that is, a multivalent metal salt and phosphoserine. In addition to the self-setting powder, component B comprises at least one hydrogen carbonate. When in contact with water or the aqueous solution the interaction of said ingredients result in a tacky and adhesive reaction mixture which contains pores.

Thus, the composition according to the present invention is preferably a ready-to-use system. The composition is injectable and can be used in a dual chamber system for simplified and fast filling of bone defects in a minimally invasive manner. With the help of a dual chamber system, this formulation can greatly facilitate clinical applications by reducing surgery time, decreasing the risk of contamination, and ensuring repeatable results.

Preferably, the phosphoserine is present in an amount from 20% to 50% by weight of the self-setting adhesive powder, preferably, 20 to 30% by weight of the self-setting adhesive powder. Such an amount of phosphoserine results in an optimal amount of pores. Furthermore, it was demonstrated that the phosphoserine content allows to tune the porosity and the curing time.

Preferably, the multivalent metal salt is present in an amount from 50 to 90% by weight of the self-setting adhesive powder, preferably, 70 to 80% by weight of the self-setting adhesive powder.

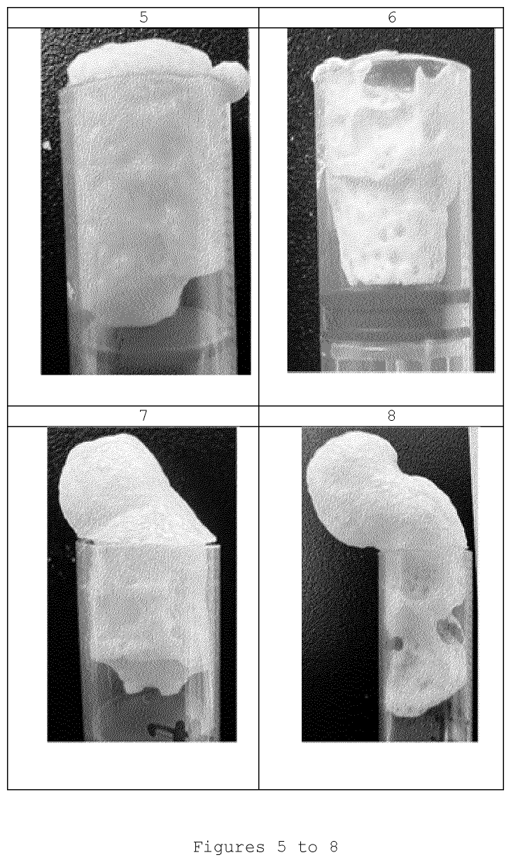

Preferably, the hydrogen carbonate is present in a concentration between 2 and 5% by weight of component B. It could be shown that said hydrogen carbonate concentration leads to an optimal result with regard to porosity and mechanical stability (see FIGS. 3 to 5).

Preferably, the water or the aqueous solution of the composition is present in an amount of up to about 35% by weight, preferably up to 25% by weight based on the combined weight of the total composition, i.e. of component A and B together. It was shown that this liquid/solid ratio resulted in an optimal setting time and compressive strength of the adhesive composition.

In one embodiment of the present invention the composition additionally comprises an acidifying agent. In order to control the pore formation, said acidifying agent is preferably part of the first component A, thus, dissolved in water or the aqueous solution. Preferably, said acidifying agent is selected from the group consisting of hydrochloric acid, ascorbic acid, citric acid, malic acid, tartaric acid, maleic acid, succinic acid, fumaric acid, acetic acid, formic acid propionic acid, their equivalent salts, and in particular trisodium citrate, tripotassium citrate, calcium citrate, magnesium citrate, ammonium citrate and iron citrate, or mixtures thereof, most preferably citric acid, sodium citrate and calcium citrate. The presence of the acidifying agent can be used to adjust the setting time. A higher concentration of the acidifying agent increases the setting time of the material during setting. Preferably, component A comprises 0.1 to 40% by weight of the acidifying agent, most preferably 5% to 25% by weight. Moreover, it has been demonstrated that the acidifying agent, or its equivalent salt, particularly sodium salt, can function as a retarder. Especially good results could be obtained with trisodium citrate.

Preferably, the hydrogen carbonate is selected from the group consisting of sodium hydrogen carbonate and ammonium hydrogen carbonate, preferably sodium hydrogen carbonate. Said hydrogen carbonates are water-soluble and biocompatible. Ammonium hydrogen carbonate has a tendency for a very fast and vigorous bubble formation. Furthermore, the size of the produced pores is larger than that produced by sodium hydrogen carbonate which leads to a larger volume grow. On the other hand, compositions comprising sodium hydrogen carbonate are easier to handle during the mixing of the ingredients and do not have a tendency to break apart upon ejection from the mixing device.

In one embodiment of the present invention multivalent metal salt contained in the composition according to the present invention comprises tetracalcium phosphate. Tetracalcium phosphate with h a comparable particle size distribution results in a slightly faster setting time and is especially preferred for large bone defects.

In another embodiment of the present invention, the multivalent metal salt contained in the composition comprises tricalcium phosphate, more preferably α-tricalcium phosphate. α-TCP is more soluble in the body's bone material which can increase its absorption rate and shortens the healing process.

Preferably, the phosphoserine in the composition according to the present invention is 1-phosphoserine. L-phosphoserine is a component of many endogenous proteins, in particular osteopontin (bone sialoprotein) and is a normal metabolite found in human biofluids. It has a high affinity for bonding to poorly crystalline apatite, suggesting it plays an important role in mineralization processes.

Preferably, the composition according to the present invention additionally comprises calcium silicate which enhances the setting reaction strength and beneficially influences bone healing and promotes osseointegration.

A further aspect relates to a kit comprising the adhesive composition according to the present invention. Said kit comprises a first compartment with a component A comprising water or an aqueous solution, and a second compartment with component B. Component B comprises a self-setting adhesive powder and at least one water-soluble hydrogen carbonate, wherein the hydrogen carbonate is present in a concentration between 1 and 8% by weight of component B. Said self-setting adhesive powder comprises at least a multivalent metal salt and phosphoserine. Said first compartment and said second compartment are physically separated from each other. The separation of the two compartments allows to provide a ready-to-use system having a long pot life.

The kit comprises preferably 10 to 35% of component A and 65 to 90% of component B, preferably 15 to 20% by weight of component A and 80 to 85% by weight of component B, whereby the total of component A and component B is 100%. It was shown that this liquid/solid ratio resulted in an optimal setting time and compressive strength of the adhesive composition.

The kit comprising the composition according to the present invention can be provided in containers selected from the group consisting of tubes, syringes, bottles, dual barrel syringes, automix systems, titurable capsules, foil packages, and combinations thereof. Such a kit allows an in situ preparation of the material directly before use.

The composition according to the present invention is preferably used in the treatment of bone defects, such as a void, a gap or a crack, so as to fill the bone defect.

Preferably, the bone defect is a large bone defect, since said bone defects are particularly difficult to treat with conventional bone graft materials. Furthermore, the composition also provides a mechanically stable scaffold for bone regrowth as well as implant stabilization. In particular, the composition according to the present invention can be used in oral or dental procedures that requires restoration, e.g., an implant placement.

According to a further aspect of the present invention the composition is used in fixing implants, in particular dental implants. Since the composition according to the present invention shows an excellent adhesive affinity for bone, metals and ceramics, the implant is provided with a good primary stability, which allows the implant to heal undisturbed. In addition, the porosity of the cured material results in a good osseointegration. Thus, the composition according to the present invention results in an excellent primary and secondary stability.

A further aspect of the present invention relates to a method for treating bone defects by applying the adhesive composition directly after mixing component A and component B to the site of the bone defect, thereby repairing the bone defect.

According to some embodiments of the invention, the method of repairing a bone defect further includes shaping the composition in the site of the bone defect, which can be done for example with a spatula or with a dedicated delivery system.

According to some embodiments of the invention, the method of repairing a bone defect further includes allowing the composition to set and cure, to thereby form a cured material.

A further aspect of the present invention relates to a method for fixing a dental implant in the site of the missing tooth root by applying the adhesive composition directly after mixing component A and component B to the site of the missing tooth or on the surface of the dental implant and placing the dental implant at the site of the missing tooth.

The cured material is formed by mixing the first component and the second component and allowing the obtained composition sufficient time to set. Initial setting may typically be achieved within 15 seconds and 90 seconds, and final setting may typically be achieved within 1 to 15 minutes. Such short setting times are acceptable since the mixing of the components can be conducted adjacent to or at the site of administration and administration follows immediately after mixing, thereby avoiding delay in surgical procedures owing to long setting times.

In one embodiment of the present invention the first component has a pH of less than 7, preferably less than 4. A pH of less than 4 results in a longer setting time of the composition. In this case, initial setting may be achieved after more than 60 seconds. Such pastes are desirable for larger, load bearing bone defects, where a longer setting time may be required. Said pastes can be manipulated, sculpted and cured in place with immediate high strength capability.

### EXAMPLES

### Material

Self-setting adhesive powder (76% α-TCP (Innotere GmbH), 24% phosphoserine (Merck))
Sodium hydrogen carbonate (NaHCO₃)
Citric acid (C₆H₈O₇)
Water (Millipore)

### Experiments

Different ratios of the listed substances were tested to evaluate their impact on the porosity of the cured material. All experiments were performed according to the following protocol:
1) Mix hydrogen carbonate with the self-setting adhesive powder (the mixing was performed in a plastic vial by shaking thoroughly)
2) Add citric acid solution or pure water
3) Mix for 10 s with a spatula in the plastic vial
4) Fill the mixture in a syringe

In the following table the different ratios can be found. The ratio of the self-setting adhesive powder and the water or the aqueous solution (with or without citric acid) was kept constant.

| | | | | Sodium | |
|---|---|---|---|---|---|
| | | | | hydrogen | |
| | | | | carbonate | |
| | Sodium | Self-setting | | in % of | |
| | hydrogen | adhesive | | component | Water |
| | carbonate | powder | Calculation | B | [μl] |
| #1 | 0.003 | 0.5 g | 0.003 g/0.503 g | 0.6 | 125 μl |
| #2 | 0.007 | 0.5 g | 0.007 g/0.507 g | 1.4 | 125 μl |
| #3 | 0.01 | 0.5 g | 0.01 g/0.51 g | 2.0 | 125 μl |
| #4 | 0.015 | 0.5 g | 0.015 g/0.515 g | 2.9 | 125 μl |
| #5 | 0.020 | 0.5 g | 0.020 g/0.52 g | 3.8 | 125 μl |
| #6 | 0.025 | 0.5 g | 0.025 g/0.525 g | 4.8 | 125 μl |
| #7 | 0.036 | 0.5 g | 0.036 g/0.536 g | 6.7 | 125 μl |
| #8 | 0.065 | 0.5 g | 0.065 g/0.565 g | 11.5 | 125 μl |

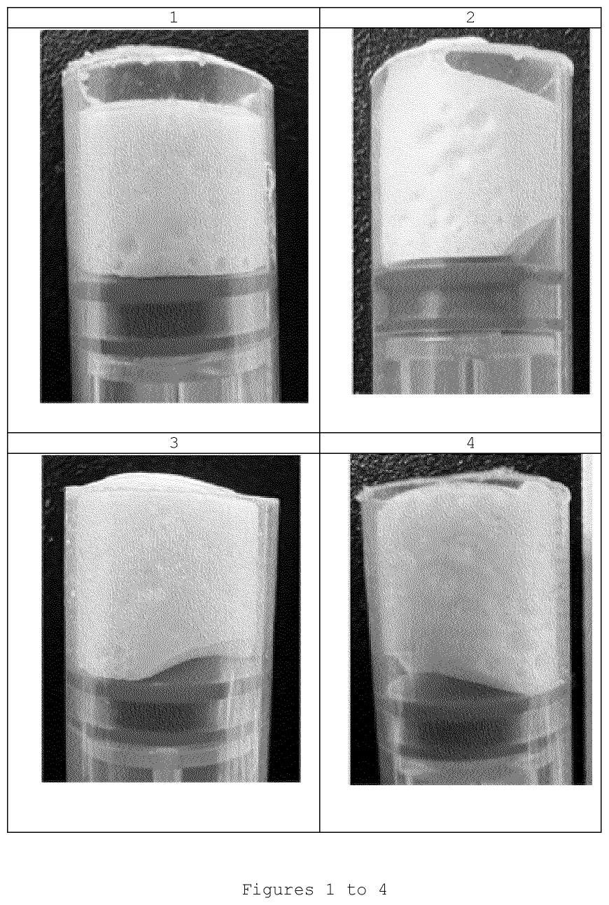

FIGS. 1 to 8 show the impact of the hydrogen carbonate on the porosity of the cured material. In sample 1 the cured material is too dense, whereas in sample 8 the pores have a negative impact on the mechanical stability. Best results are obtained with samples 3, 4 and 5.

### Example 2: Infiltration of Blood

Samples (3% [w/w]) are prepared with/without porosities and a suitable shape is prepared by molding in 3 ml syringes. The weight of the samples is measured and recorded (FIG. 9A (with hydrogen carbonate and 9B without hydrogen carbonate).

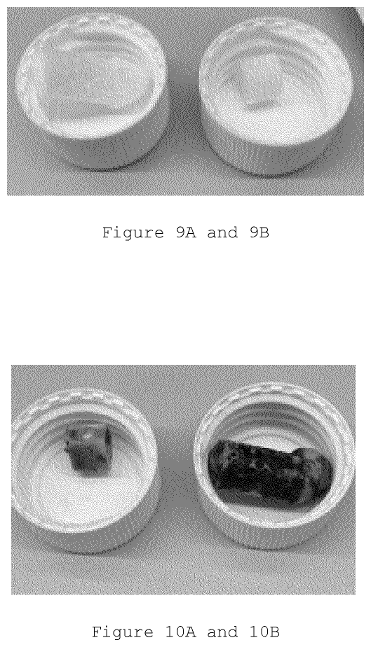

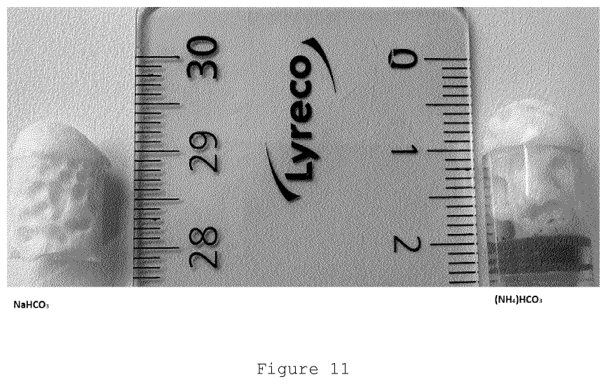

Afterwards, the samples are submerged into whole blood for 10 minutes. Samples are retrieved from the blood (excess blood is allowed to run off the samples) and the weight is recorded (FIG. 10A (without hydrogen carbonate) and 10B (with hydrogen carbonate)).

| | | Weight after | |
|---|---|---|---|
| | Weight before | immersion | Weight |
| | immersion [g] | [g] | gain [%] |
| Non-porous | 0.7233 | 0.8007 | 10.70 |
| (Comparative | | | |
| example) | | | |
| Porous | 0.7088 | 1.0631 | 49.99 |
| (according | | | |
| to the | | | |
| present | | | |
| invention) | | | |

A weight gain of 10.70% was measured for the dense material compared to 49.99% for the material according to the present invention.

### Example 3: Acidifying Agent

Citric acid solutions of 15 and 30% [w/v] were prepared and the pH of the solutions determined. The pH value of the two solutions is comparable:

$$15 \% [ w / v ] \to pH = 3.3$$

$$30 \% [ w / v ] \to pH = 3.1$$

It was tested whether the use of different concentrations of citric acid has an effect on the composition.

The bubble formation is, compared to experiments with no citric acid, slow and appears to continue for a longer period of time.

It was found that a lower concentration (15% w/v) of citric acid can influence setting-time and compressive strength of the final product.

### Example 4: Comparison Hydrogen Carbonate and Monocarbonate

Self-setting adhesive powder with hydrogen carbonate at 3% [w/w] was prepared.

Self-setting adhesive powder with monocarbonate at 3% [w/w] was prepared.

The following observations were made:

The use of NaHCO₃ resulted in a larger volume growth (the resulting sample was ˜57% larger), compared to Na₂CO₃.

The material prepared using NaHCO₃ resulted in larger pores, compared to the material prepared using Na₂CO₃. The material prepared using Na₂CO₃ seemed not to be suitable for a use in larger defects in vivo.

### Example 5: Ammonium Hydrogen Carbonate

An equimolar formulation corresponding to the 3% [w/w] NaHCO₃ formulation was prepared.

For (NH₄)HCO₃ bubble formation was very fast/vigorous compared to sodium hydrogen carbonate at the same concentration. In fact, it was so fast that the mixing was more complicated compared to e.g. NaHCO₃.

The volume growth that was seen for (NH₄)HCO₃, resulted in an approximate 7% larger volume than for the NaHCO₃ sample.

The bubbles produced by (NH₄)HCO₃ were visibly larger than those produced by NaHCO₃(FIG. 11).

Upon ejection from the syringe, the (NH₄)HCO₃ sample had a tendency to break apart.

### Example 6: Comparison α-TCP Vs TTCP

| Formulation | | | Phosphoserine |
|---|---|---|---|
| name | α-TCP | TTCP | ratio [w/w] |
| Alpha12 | 88% | | 12% |
| Tetra12 | | 88% | 12% |
| Alpha24 | 76% | | 24% |
| Tetra24 | | 76% | 24% |
| Alpha36 | 64% | | 36% |
| Tetra36 | | 64% | 36% |

For each experiment, 0.5 g of the self-setting powder and 15 mg sodium bicarbonate (3% w/w) were mixed with 0.125 ml water.

### Example 6a: Comparison of Alpha24 and Tetra24

15 mg (corresponds to 3% w/w of the powder) of sodium bicarbonate was added to the powder before mixing for 10 s with water.

The following observations were made:
similar pore formation for both TCP's,
similar overall volume/porosity for both TCP's,
TTCP sets appr. 25% faster compared to α-TCP,
overall volume gain/final porosity is higher for α-TCP, presumably due to the slower setting, since there is more time for gas expansion/pore formation,
TTCP is slightly more brittle.

### Example 6b: Influence of Phosphoserine (PS) Content

3% w/w Sodium bicarbonate was added to each of the powder formulations before mixing for 10 s with water.

The following observations were made:
the higher the PS content, the higher the final volume/porosity of the samples,
for 12% PS the pore formation is minimal,
the mixture was of high viscosity (clay-like) during the whole mixing process. This might be one of the reasons why pore formation was hindered during the process,
for 36% PS the overall volume/porosity appeared to be higher for α-TCP compared to TTCP. The faster setting time of TTCP could be the reason for that.

### Example 6c: Influence of Citric Acid

Alpha12, Alpha24, Tetra12 and Tetra24 with 15% citric acid were compared to their analogues without citric acid.

The following observations were made:
for both 12% PS formulations, adding citric acid makes them behave more like the 24% PS formulations without citric acid. In terms of viscosity as well as for the final volume/pore formation (neglectable pore formation of the 12% PS+water samples, could be reversed by using citric acid),
also for both 24% formulations, adding citric acid lead to a larger overall volume/pore formation. That was actually comparable to the corresponding 36% PS samples without citric acid.

### Example 6d: Influence of Trisodiumcitrate

Same formulations were used as for experiment 3 but the groups with water were exchanged with trisodium citrate.

The following observations were made:
the overall final volume/porosity appears to be comparable between citric acid and trisodium citrate,
however, the samples with citric acid cured faster compared to the samples with trisodium citrate. Trisodium citrate seems to have stronger retardant properties.
regarding the choice of retardant, no differences could be observed between Alpha- and Tetra-TCP.

1. A two-component composition comprising
a first component A comprising water or an aqueous solution, and
a second component B comprising
1) a self-setting adhesive powder comprising at least a multivalent metal salt and phosphoserine, and
2) at least one hydrogen carbonate,
wherein the hydrogen carbonate is present in a concentration between 1 and 8% by weight of component B.

2. The composition according to claim 1, wherein the hydrogen carbonate is present in a concentration between 2 and 5% by weight of component B.

3. The composition according to claim 1, additionally comprising an acidifying agent selected from the group consisting of hydrochloric acid, ascorbic acid, citric acid, malic acid, tartaric acid, maleic acid, succinic acid, fumaric acid, acetic acid, formic acid, propionic acid, their corresponding salts or mixtures thereof.

4. The composition according to claim 3, wherein the acidifying agent is part of the first component A.

5. The composition according to claim 4, wherein component A comprises 0.1 to 40% by weight of the acidifying agent.

6. The composition according to claim 1, wherein the hydrogen carbonate is selected from the group consisting of sodium hydrogen carbonate and ammonium hydrogen carbonate.

7. The composition according to claim 1, wherein the multivalent metal salt comprises tetracalcium phosphate.

8. The composition according to claim 1, wherein the multivalent metal salt comprises tricalcium phosphate.

9. The composition according to claim 1, wherein the self-setting adhesive powder of component B additionally comprises calcium silicate.

10. A kit for preparing a composition according to claim 1, comprising
a first compartment with a component A comprising water or an aqueous solution, and
a second compartment with component B comprising
1) a self-setting adhesive powder comprising at least a multivalent metal salt and phosphoserine,
2) at least one hydrogen carbonate,
wherein the hydrogen carbonate is present in a concentration between 1 and 8% by weight of component B, and wherein the first compartment and the second compartment are physically separated from each other.

11. The kit according to claim 10, comprising 10 to 35% of component A and 65 to 90% of component B, whereby the total of component A and component B is 100%.

12. Composition according to claim 1 for use to restore or repair bone.

13. Composition according to claim 12 for use in the treatment of bone defects.

14. Composition according to claim 1 for use to fix implants.

15. Composition according to claim 14 for use to fix a dental implant to bone tissue or soft tissue.
